(19) United States
(12) Patent Application Publication
(10) Pub. No.: US 2023/0290485 A1
(43) Pub. Date: Sep. 14, 2023

(54) ARTIFICIAL INTELLIGENCE PRIORITIZATION OF ABNORMAL RADIOLOGY SCANS

(71) Applicant: International Business Machines Corporation, Armonk, NY (US)

(72) Inventors: Sumaiya Fairuz, Hilliard, OH (US); Emanuel Ibarra, Austin, TX (US); Candyce Nicole Jessamy, Austin, TX (US); Alexandra Huisgen Serrano, Barcelona (ES); Laura Marshall, Reading (GB)

(21) Appl. No.: 17/740,393

(22) Filed: May 10, 2022

(30) Foreign Application Priority Data
Mar. 9, 2022 (EP) 22382220.6

(51) Int. Cl. G16H 30/20; G16H 15/00; G16H 50/20; G16H 50/30

(52) U.S. Cl. CPC G16H 30/20; G16H 15/00; G16H 50/20; G16H 50/30

(57) ABSTRACT

An approach for prioritizing the review of medial image scans in a medical record review program may be provided. The approach may include analyzing one or more medical image, where each image scan is associated with a patient from a plurality of patients. The analysis of the medical image scan may be based on a machine learning algorithm. The approach may also include identifying an abnormal condition(s) from the analyzed medical scans, where the identified abnormal condition(s) is based on the analysis. The approach may further include prioritizing the identified abnormal condition(s) if the abnormal condition is an urgent condition. Additionally, the approach may include presenting a scaled down version of the prioritized medical scan within a mini viewer window within a medical record review program.

### BACKGROUND OF THE INVENTION

The present invention relates generally to the field of machine learning and artificial intelligence, more specifically to identification and review prioritization of radiology scans with abnormal conditions.

Radiologists read hundreds of patient cases per day and each individual case can contain numerous medical scans. The scans can be from numerous sources, such as x-ray images, medical resonance imaging (“MRI”), computerized tomography (“CT”) scan, computerized axial tomography (“CAT”), positron emission tomography (“PET”) scan, and ultrasound scans. Algorithms have been developed to identify medical conditions from various medical scans with differing levels of confidence. For example, convolutional neural networks have been utilized to identify abnormalities in CT and CAT scans.

Computerized systems for patient electronic health records (“EHR”) management allow medical professionals efficient and easy access to a multitude of patient data. These electronic health records have provided an environment where patient data can be cross checked to ensure safety and allow for prioritization of treatment of individuals within a medical facility.

### SUMMARY

Embodiments of the present disclosure include a computer-implemented method computer system, and computer program product for prioritizing medical scan review within an electronic medical record review system. The embodiments may include analyzing one or more medical scans each associated with a patient from a plurality of patients, based on a machine learning algorithm. Additionally, some embodiments may include identifying at least one abnormal condition on the one or more medical scans, based at least in part on the analysis. Responsive to identifying at least one abnormal condition, some embodiments may include, prioritizing the at least one identified abnormal condition, wherein the abnormal condition is an urgent condition, based at least in part on the machine learning algorithm. Some embodiments may also include presenting a scaled down version of the medical scan in a mini viewer window within the medical record review system.

### BRIEF DESCRIPTION OF THE DRAWINGS

FIG. 1 is a functional block diagram of a medical image review prioritization system, generally designated 100, in accordance with an embodiment of the present invention.

FIG. 2 is a block diagram of patient medical scan prioritization engine 104, in accordance with an embodiment of the present invention.

FIG. 3 is flowchart of a method for generating an explanation for change risk classification 300, in accordance with an embodiment of the present invention.

FIG. 4 is a functional block diagram of an exemplary computing system 10 within a medical image review prioritization system, in accordance with an embodiment of the present invention.

FIG. 5 is a diagram depicting a cloud computing environment 50, in accordance with an embodiment of the present invention.

FIG. 6 is a functional block diagram depicting abstraction model layers, in accordance with an embodiment of the present invention.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Embodiments presented herein recognize the need for identifying and prioritizing the review of medical scans from the most urgent cases. In many cases, radiologists and medical specialists tasked with reviewing medical scans do not have an efficient way of reviewing or knowing which cases within a medical scan reviewing program require quick review and diagnosis based on the medical scan. While some computer aided detection mechanism and models have been developed, there is not currently a way for prioritizing review of medical scans. For example, some medical scan review programs present medical image scans for review in chronological order, while others may present the medical scan images in alphabetical order based on patient name or the attending physician's name. What is required is a system which can analyze the medical image scans and prioritize the scans, placing the scans at the forefront of the reviewing radiologist or specialists work queue or in a separate urgent case list.

In an embodiment of the invention, a machine learning algorithm can analyze a medical image scan. A medical scan can be an X-ray, MRI, CT scan, CAT scan, tomography (“PET”) scan, and ultrasound scans. It should be understood, the terms medical scan, medical image scan and medical image, will be used throughout this description interchangeably and should be afforded the same meaning. Based on the analysis of the medical image scan, the machine learning algorithm may identify one or more abnormal conditions in the medical image scan. If the machine learning algorithm has identified an abnormal condition within the scan, the machine learning algorithm can prioritize the medical image scan. Further, the prioritized scan can be presented within a mini-viewer or urgent case list within a medical image scan review program.

In another embodiment of the invention, the machine learning algorithm may include one or more predictive models trained to identify abnormal conditions within a medical image scan. For example, a predictive model may be trained to identify cancerous growths in a medical scan. It should be noted, a model may be trained to identify one or more type of cancerous growth in a specific organ. Further, the machine learning model may have one or more predictive models which may feed into a downstream model that may receive more than one scan to more accurately predict an abnormal condition. For example, a model may be trained to identify soft tissue damage (e.g., muscle ruptures, tendon tears, etc.) while a second model may be trained to identify damage to internal organs (e.g., internal bleeding, kidney stones, lesions, cysts, etc.) the downstream predictive model may receive predictions from these two models to more accurately highlight or identify an abnormal condition.

In an embodiment, the identified abnormal condition may be highlighted via a box or circle surrounding the abnormal condition and/or presented with a short description of what the identified abnormal condition may be. For example, an ultrasound scan of the liver may contain lesions. The predictive model may identify the lesions and highlight the area of the scan containing the lesions with a circle or a shape which fits the lesions. Further, the predictive model may include a label of the lesion or even the type of lesion. It should be noted that a confidence score may also be presented in the identification. As in the immediately preceding example of the lesion, identification may have a notation as follows, “liver lesion—87% confidence.”

In another embodiment of the invention, the machine learning model may have a prioritization model. The prioritization model may utilize the identified abnormal condition and additional factors to prioritize or rank the urgency with which the medical image scan should be reviewed. For example, if it is identified via an MRI scan of the colon at 95% confidence that a patient has stage 4 colon cancer, that may receive a high prioritization. Further, additional factors may be included in the prioritization model that can be found within the patient's EHR, such as past medical history of illness or disease (e.g., past cancer or was previously in remission for the same type of cancer), the age of the patient, condition of the patient at time of scan (e.g., stable, non-stable, critical, etc.), family history.

In another embodiment of the invention, the prioritization model may rank multiple scans of different patients for review (i.e., triage). In this embodiment, each scan may contribute to an overall prioritization score or the scan depending on the abnormal condition may individually receive a prioritization score. The prioritization score may be on a scale of 0-100 where 100 is the highest priority. An abnormal condition itself may not trigger an immediate prioritization of review, rather, additional factors (as described above) can contribute to the score ranking.

In an embodiment, the prioritization model can trigger the presentation within a mini-viewer window or urgent list and preloading of the medical scans associated with a patient, specifically the scan that has been identified as urgent. In an embodiment, there can be a prioritization score threshold in which the prioritization score of the scan or patient triggers presentation within the mini-viewer. It should be understood that the terms mini-viewer and urgent list will be used interchangeably thorough out this description and should be afforded the same meaning. The threshold for presentation within the mini-viewer can be dynamic or static. Factors such as the number of cases in the reviewing specialist's workload, number of identified abnormal scans, and/or frequency of new scans (e.g., if a large traffic accident, industrial accident, or weather catastrophe were to occur.) Further, the number of cases (1, 2, n . . . n+1) that can be included in the mini-viewer can change depending on the same factors as described above.

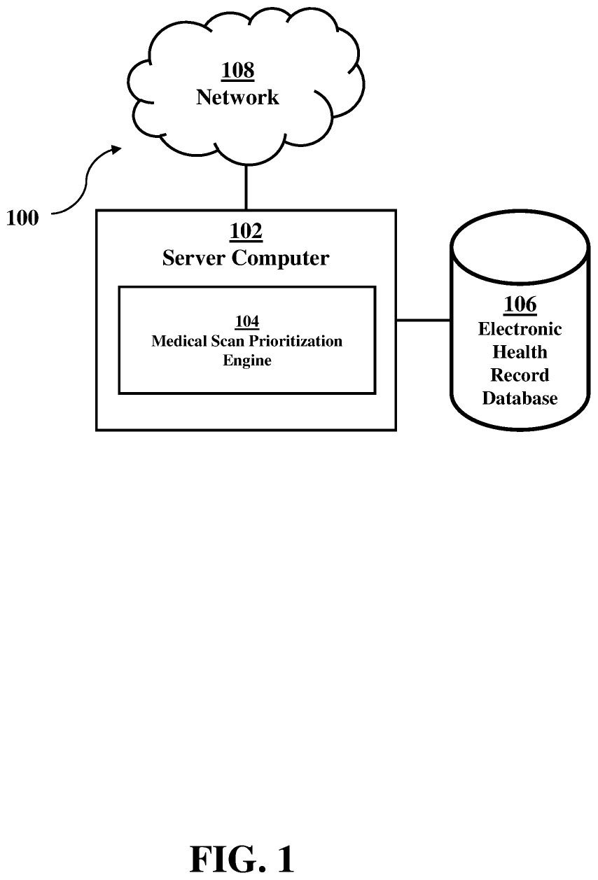

FIG. 1 is a functional block diagram of medical scan review prioritization system 100. Medical scan review prioritization system 100 comprises medical scan prioritization engine 104 operational on server 102, and electronic health record database 106. Also shown in FIG. 1 is network 108 to allow for communication between server 102 and other computing devices (not shown).

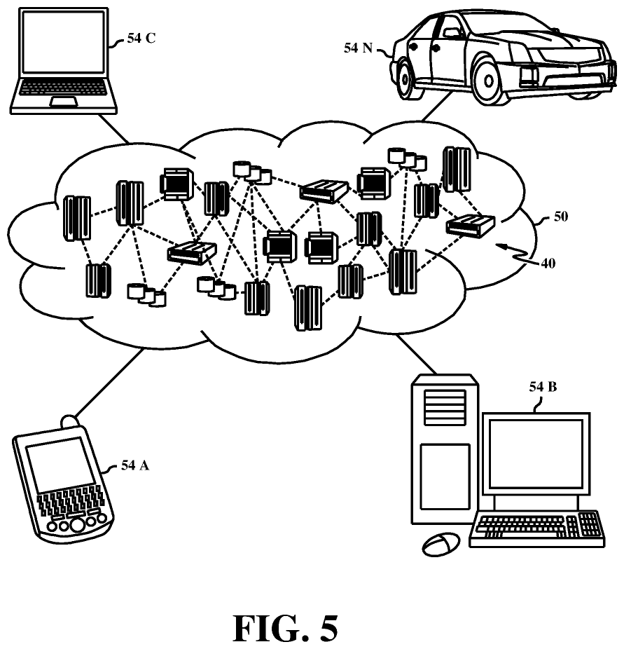

Server 102 can be a standalone computing device, a management server, a web server, a mobile computing device, or any other electronic device or computing system capable of receiving, sending, and processing data. In some embodiments, server 102 can represent a server computing system utilizing multiple computers as a server system such as in cloud computing environment 50 (depicted in FIG. 5). In an embodiment, server 102 can represent a computing system utilizing clustered computers and components (e.g., database server computers, application server computers, etc.) that act as a single pool of seamless resources when accessed within medical scan review prioritization system 100. In another embodiment, server 102 can be a laptop computer, a tablet computer, a netbook computer, a personal computer, a desktop computer, or any programmable electronic device or combination of programmable electronic devices capable of executing machine readable program instructions and communicating with each other and other computing devices (not depicted) within medical scan review prioritization system 100 via network 108. It should be noted, while only server 102 is shown in FIG. 1, in other embodiments, multiple servers or other computing devices can be present within medical scan review prioritization system 100.

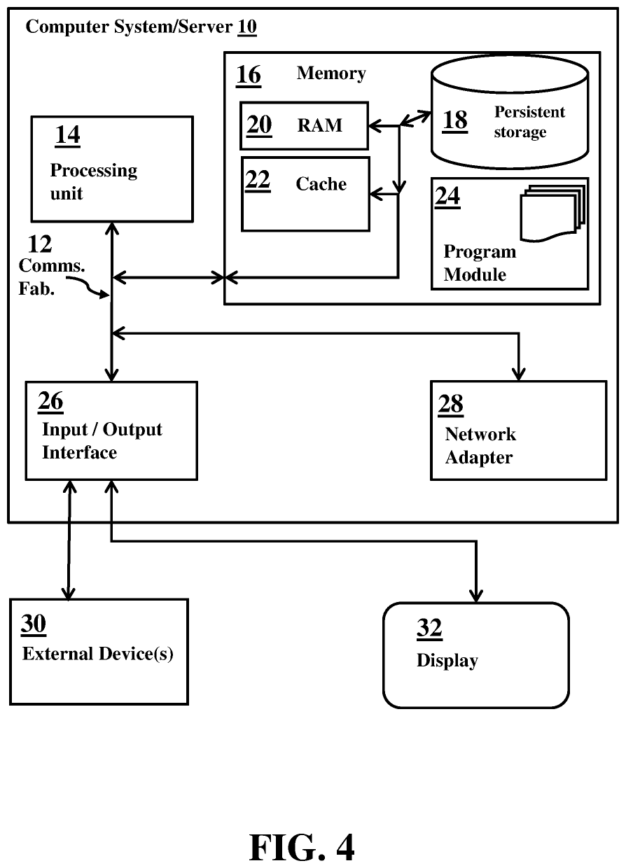

Server 102 may include components as depicted and described in further detail with respect to computer system 10 in FIG. 4. Server 102 may include components as depicted and described in further detail with respect to cloud computing node 40 of cloud computing environment 50 in FIG. 5.

Network 108 can be a telecommunications network, a local area network (LAN), a wide area network (WAN), such as the Internet, or a combination of the three, and can include wired, wireless, or fiber optic connections. Network 108 may include one or more wired and/or wireless networks that are capable of receiving and transmitting data, voice, and/or video signals, including multimedia signals that include voice, data, and video information. In general, network 108 can be any combination of connections and protocols that will support communications between server 102, and external computing devices (not shown) within medical scan review prioritization system 100.

Medical scan prioritization engine 104 is a computer program capable of performing at least the following operations: (i) analyzing a medical scan, (ii) identifying abnormal conditions within the medical scan, (iii) determining whether review of the medical scan is urgent, and (iv) presenting the medical image scan in an urgent review list within a medical scan review user interface (described in further detail below). Further, in an embodiment, medical scan prioritization engine 104 may be comprised of a machine learning model, capable of automatically updating itself based on feedback from a medical professional, regarding the determination of whether an abnormal condition is present, and whether an abnormal condition is urgent, as well as and whether an abnormal condition is in fact an abnormal condition, and not a false positive or false negative.

In an embodiment, medical scan prioritization engine 104 or various computer modules operational on medical scan prioritization engine 104 (described in more detail below), may be configured to access various data sources, such as electronic health record database 106 (described further below) that may include personal data, content, contextual data, or information that a patient does not want to be processed. Personal data includes personally identifying information or sensitive personal information (e.g., medical/health records) as well as patient information, such as location tracking or geolocation information. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal data. In an embodiment, medical scan prioritization engine 104 enables the authorized and secure processing of personal data. In an embodiment, custom thumbnail program provides informed consent, with notice of the collection of personal data, allowing the user to opt in or opt out of processing personal data. Consent can take several forms. Opt-in consent can impose on the patient to take an affirmative action before personal data is processed. Alternatively, opt-out consent can impose on the patient to take an affirmative action to prevent the processing of personal data before personal data is processed. In an embodiment, medical scan prioritization engine 104 provides information regarding personal data and the nature (e.g., type, scope, purpose, duration, etc.) of the processing. In an embodiment, medical scan prioritization engine 104 provides a patient with copies of stored personal data. In an embodiment, medical scan prioritization engine 104 allows for the correction or completion of incorrect or incomplete personal data. In an embodiment, medical scan prioritization engine 104 allows for the immediate deletion of personal data.

Electronic health record database 106 is a database that contains electronic health records of patients. For example, health records can contain patient data including medical image scans, medical test results (complete blood profiles, neurology test results, etc.), medical professional reports (e.g., physician's notes, radiology reports, etc.), and self-reported injury, illness, family history (e.g., diabetes, cancer, addictive tendencies, etc.), and medical conditions. The medical reports and self-reported medical information within the health records can be in human readable format, such as human handwriting, or type written. It may contain scans of physical x-rays or other medical image scans on film or electronic versions of medical scans. Further it may contain digitized results of medical tests.

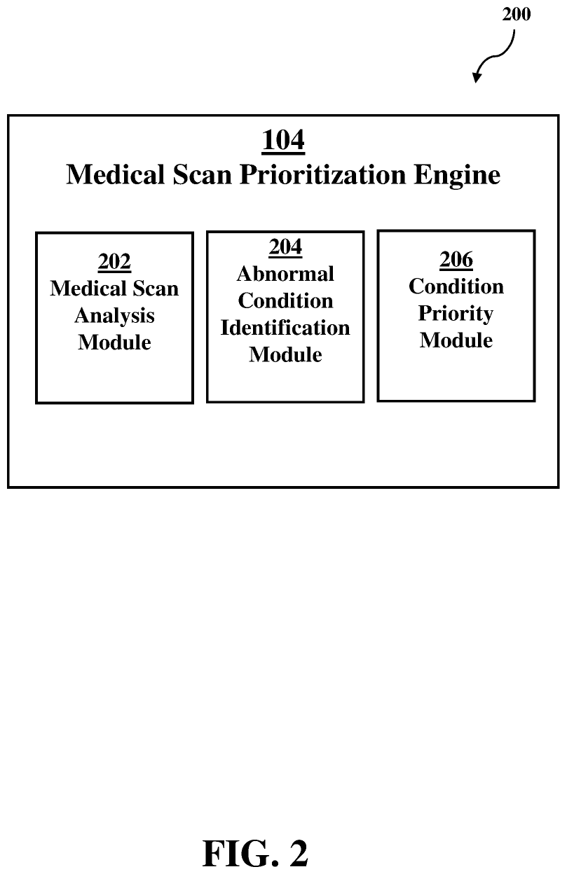

FIG. 2 is block diagram 200 of medical scan prioritization engine 104. Medical scan prioritization engine 104 can be comprised of medical scan analysis module 202, abnormal condition identification module 204, and condition priority module 206.

Medical scan analysis module 202 is a computer module that can be configured to receive medical image scans and identify portions of the medical image scans that may be abnormal conditions. In an embodiment, medical scan analysis module 202 can utilize a machine learning model for computer aided detection. For example, the machine learning model can be a deep neural network, such as a convolutional neural network, that has been trained to analyze medical scan images for abnormal conditions.

In an embodiment, medical scan analysis module 202 can analyze a medical image scan, such as an MRI of the liver. In this example, medical scan analysis module 202 can be a convolutional neural network and condense the MRI scan of the liver into convolutions (i.e., smaller sections) and reanalyzed to remake the identify abnormal conditions based on the convolutions. While multiple scans of slices of the liver would be taken, in this example only one slice will be discussed for simplicity's sake. The pixels with the abnormal condition can be highlighted and an area of abnormal pixels can be shown highlighted.

Abnormal condition identification module 204 is a computer module that can be configured to receive an analysis from medical scan analysis module 202 and identify an abnormal condition from the highlighted sections of the medical scan. In an embodiment, abnormal condition identification module 204 can be a machine learning algorithm trained to identify one or more abnormal conditions based on shape, size, color, location within an organ or the like. Further, abnormal condition identification module 204 may have multiple machine learning models or artificial intelligence loops which can identify multiple abnormal conditions within a medical scan. For example, in a three dimensional MRI scan of a patients lungs, one model may identify a cancerous tumor, from a mass of cells highlighted by medical scan analysis module 202, while another machine learning model may identify scarring of alveoli tissue highlighted by medical scan analysis module 202.

In an embodiment, abnormal condition identification module 204, can provide a confidence score of the identified abnormal condition. For example, a small, circular or normal shaped mass might be highlighted by medical scan analysis module 202. Due to the size of the mass abnormal condition identification module 204 could predict a benign tumor with a 24% confidence. While in another scan, a large irregularly shaped mass could be identified as a mast cell tumor with 88% confidence. Multiple factors can contribute to the confidence score. For example, the location of the highlighted area within the scan (where the orientation of the organs or body is known), the clarity of the scan, or details relating to the patient (e.g., age, medical history, etc).

In another embodiment, abnormal condition identification module 204 can ignore highlighted pixels in a medical scan. For example, medical scan analysis module 202 may highlight pixels within an image that do not represent an abnormal condition. In this instance, there is no abnormal condition. Abnormal condition identification module 204 can either ignore the highlighted pixels or label them as null. This may be the case where a model associated with abnormal condition identification module 204 does not associate the highlighted pixels with an abnormal condition it was trained to identify. In another example, due to an abnormality with the patient or medical scan, it could be a normal scan and the highlighted pixels could be a false highlight. In this case, abnormal condition identification module 204 may label the highlighted pixels as normal, rather than label them with a specific condition.

Condition priority module 206 is a computer module that can be configured to prioritize identified medical conditions and present the medical scan in a mini viewer within a medical scan review program. In an embodiment, condition priority module 206 may receive the identified abnormal condition and scan from abnormal condition identification module 204. Condition priority module 206 can utilize a machine learning algorithm to determine whether the scan with the identified abnormal condition should receive priority review or is considered urgent. In some embodiments, condition priority module 206 can place the medical scan in order of priority review. For example, medical scans can be from an electronic health record from electronic health record database 106 for a patient. Condition priority module 206 may scan the electronic health record for pertinent data which can include past medical conditions associated with the area consistent with the medical scan, age, past diagnosis. It should be noted, some abnormal conditions may simply qualify as an urgent condition that required immediate review. These will typically be life threatening conditions such as aggressive forms of cancer, traumatic injuries such as a fractured skull or compound fracture, and the like.

In an embodiment, condition priority module 206 can generate a priority score based on multiple factors. For example, the identified abnormal condition may have a base score or factor associated with it in determining the score (e.g., benign tumor in a muscle 0.3, metastasized tumor in brain stem 3.4) The score can be calculated based on the patients age and general health conditions accessed through electronic health record database 106. The score can be calculated and run through a softmax operation to provide a score between 0 and 100.

In an embodiment, condition priority module 206 can present the prioritized medical scans in a mini viewer within a medical scan review program. In some embodiments, all medical scans above a threshold (e.g., 70) can be placed within the viewer. The threshold can be dynamic, or static based on factors such as number of patients, average priority score, or number of logged in specialists reviewing medical scans. For example, the number of medical scans within the mini viewer can be limited to a specific number, with the cases with the highest priority score presented in the mini viewer. In another example, all medical scans with a priority score above 70 can be placed in the mini viewer. In the instant case, the mini viewer can be a window with a method of viewing (e.g., scrolling) cases if there are more than can be shown on a display screen. In another example, if no scores are above a threshold, no cases may be placed within the mini-viewer. A dynamic threshold could be utilized in this case and place a number of cases within the mini viewer. It should be noted, in an embodiment, the length of time a medical scan has been in the work queue of the specialist can be a factor for prioritization of review.

In an embodiment, condition priority module 206 can allow for the identification of abnormal conditions to populate the medical scan review program without presenting any cases within the mini-viewer. For example, when no medical scans are above the priority rating threshold, any abnormal conditions identified by abnormal condition identification module 204 will still be associated with the medical scan. Condition priority module 206 can prioritize and sort the main workload review queue for the specialist. Condition priority module 206 can be customizable and automatically any present associated scans deemed urgent by the specialist or set by a medical scan program administrator.

In another embodiment, condition priority module 206 can allow for a specialist to flag a medical scan within the mini viewer for further review by another specialist. In this instance, condition priority module 206 can dissociate a medical scan priority score and automatically place the flagged medical image in the top of the mini viewer for another specialist, thus providing an override to the priority review score by a specialist.

In an embodiment, condition priority module 206 can scale down the medical image allowing for a thumbnail or scaled down (e.g., ½ size or ⅓ size) sized view of the medical scan within a mini viewer window. In another aspect, condition priority module 206 can actively manage the mini viewer priority list and preload all of the prioritized medical scans within the mini viewer or the prioritized medical scans that are visible within the mini viewer on the display. This can ensure faster load times of the priority scans when a specialist clicks to review the medical scan.

In another aspect of the invention, condition priority module 206 can receive feedback from the specialist from input fields associated with the specialist report (e.g., radiology reports). In this embodiment, condition priority module 206 can receive the report. For example, in a medical scan prioritized with a liver lesion, the radiology report may include language confirming the liver lesion and describing the size and maturity of the lesion and any additional scarring. Condition priority module 206 can be equipped with a natural language processing model that can recognize the language used by the radiologist. Condition priority module 206 can proceed to provide feedback to abnormal condition identification module 204 which can fine tune the model responsible for abnormal condition for liver lesions.

Further, in a scenario where the incorrect condition is identified (e.g., a malignant tumor is diagnosed by the specialist as a cyst), condition priority module 206 can provide the feedback from the report to the abnormal condition identification module 204, allowing for the fine tuning of the model and the resulting confidence score. Thus, as multiple iterations of feedback occur, condition priority module 206 can assist the specialist in identification of abnormal urgent conditions by fine tuning and providing increased rewards to the model (e.g., adjusting the loss function of a model). Additionally, the feedback can include whether or not the condition would be considered urgent and if the case should be considered a priority. For example, there may be a check box for urgent or not urgent. Further, if a specialist hovers a mouse pointer over a case presented in the mini viewer to see the scaled down medical scan and does not choose to review the case, this would indicate the priority score for the medical scan is too high. This feedback can be included in the machine learning algorithm of electronic health record database 106 and the algorithm can be fine-tuned to reduce similar priority scores of cases in the future.

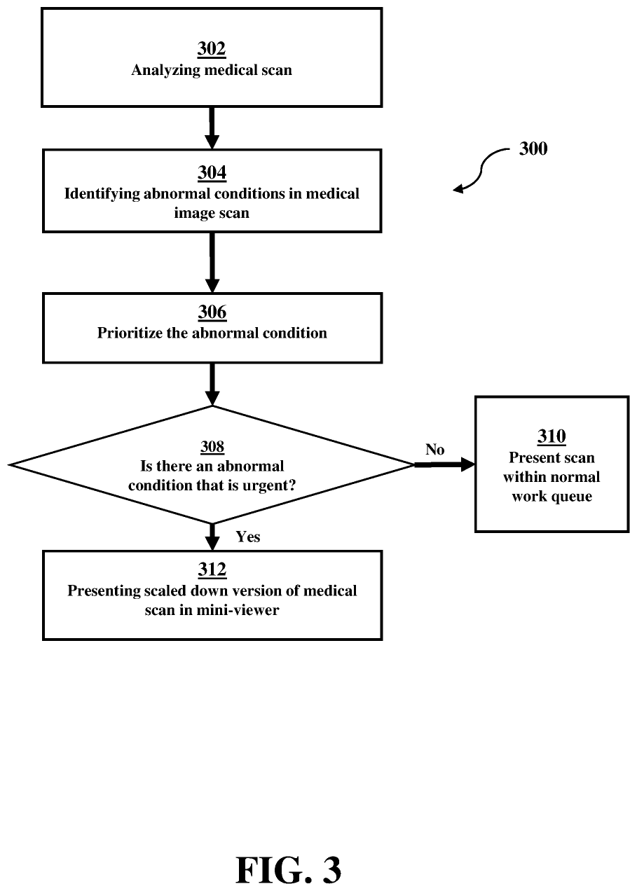

FIG. 3 is a flowchart depicting method 300 for prioritizing the review of medical scans in a medical record review system, in accordance with an embodiment of the present invention.

At step 302, medical scan analysis module 202 can analyze a medical scan associated with a patient's EHR from EHR database 106. In an embodiment, medical scan analysis module 202 can be operational within medical scan prioritization engine 104 and have access to EHR database 106. The medical scans can be received by or retrieved by medical scan analysis module 202. The medical scan can be analyzed by a computer assisted detection algorithm or a detection model such as a neural network, deep neural network, convolutional neural network or the like. In an aspect, the analysis can highlight or note pixels that might correspond to an abnormal condition. If multiple adjacent pixels are highlighted, an outline can be made of all the pixels allowing for easy viewing of a reviewing specialist.

At step 304, abnormal condition identification module 204 can receive one or more analyzed medical scans and identify one or more abnormal conditions based on the analysis. For example, abnormal condition identification module 204 can be a model trained to identify conditions associated with a specific medical scan and/or a specific organ/body system (e.g., major joints, the brain, pulmonary system, cardiac system). In one aspect of the present invention, abnormal condition identification module 204 can determine or generate a confidence score for the identified abnormal condition within the medical scan. For example, in the same highlighted section of an analyzed scan multiple abnormal conditions may be identified. In this instance, the abnormal condition with the highest confidence score can be labelled on the highlighted area. In another instance, if a confidence score of an abnormal condition is not above a threshold (e.g., 50%), no abnormal condition will be associated with the highlighted pixels of the medical scan.

At step 306, condition priority module 206 can prioritize the identified abnormal condition. In an embodiment, condition priority module 206 can have a base score or factor for every identifiable abnormal condition. Condition priority module 206 can access further details of the patient from which the scan was taken and determine a priority score. For example, the overall condition (e.g., good, fair, serious, or critical) of a patient may be in the most recent physician's report (age, family history, medical conditions) and/or live data from the medical instruments (electrocardiogram, blood pressure, pulse) monitoring the patient may be considered in the determination of the priority score. It should be noted, condition priority module 206 can utilize one or more machine learning algorithms or machine learning models (e.g., deep neural networks, graph networks, etc.) to determine the condition priority score. It should also be noted, if more than one abnormal condition is found within a scan, both conditions can be included in the determination of the priority score, either by adding the two scores together or multiplying the scores by a factor.

At decision step 308, condition priority module 206 can determine if the identified abnormal condition or medical scan qualifies as urgent. For example, if the priority score is above a predetermined or dynamically set threshold, condition priority module 206 can determine if the identified abnormal condition qualifies as an urgent condition. In an embodiment, condition priority module 206 can dynamically set the threshold to determine if an abnormal condition qualifies as abnormal. If condition priority module 206 determines that an abnormal condition or scan qualifies as urgent, the process proceeds to step 312. If condition priority module 206 determined that an abnormal condition does not qualify as urgent, the process proceeds to step 310.

At step 310, if condition priority module 206 determines that an abnormal condition or scan is not urgent, condition priority module 206 can place the scan in the normal workload queue of the specialist or in the normal workload review queue of the medical scan review program to be assigned to the next available specialist to review.

At step 312, if condition priority module 206 determines that an abnormal condition or scan is urgent, condition prioritization module 206 can present the scan with the urgent abnormal condition within a mini viewer of the medical scan review program. The mini-viewer is for medical scans that have been deemed urgent and should be reviewed by a specialist before other cases due to the complexity of the condition or the severity of the condition. In an embodiment, condition prioritization can present one more or scans in the mini-viewer in a thumbnail allowing the specialist to hover the selection tool (e.g., mouse, or light touch for a touchscreen display) allowing a scaled down format image of the scan to be seen on the display. Further, in an aspect of the present invention upon presenting the scan in the mini-viewer, condition prioritization module 206 can pre-load the full size image of the medical scan, for quick loading upon selection for review by a specialist. It should be noted, the mini-viewer can be continuously populated by condition prioritization module 206 as more scans are processed by medical scan prioritization engine 104 or additional real time patient data is uploaded and processed by medical scan prioritization engine 104.

In an embodiment, medical scan prioritization engine 104 can simultaneously perform all actions of the modules operational within it. This can result in the seamless presentation of medical scans deemed urgent within the mini-viewer and non-urgent scans in the normal work queue of the specialist within the medical scan review program. It should be noted, inputs from a specialist can be used in a continual feedback loop to continuously train and refine the machine learning models and artificial intelligence models equipped on the modules within medical scan prioritization engine 104, with one goal of to be the fast and efficient review of medical scan with few to no misdiagnosis of medical scans, due to the machine learning models and artificial intelligence models learning the tendencies and knowledge of the specialist.

FIG. 4 depicts computer system 10, an example computer system representative of server 102, or any other computing device within an embodiment of the present invention. Computer system 10 includes communications fabric 12, which provides communications between processing unit 14, memory 16, persistent storage 18, network adaptor 28, and input/output (I/O) interface(s) 26. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses or a crossbar switch.

Memory 16 and persistent storage 18 are computer readable storage media. In this embodiment, memory 16 includes persistent storage 18, random access memory (RAM) 20, cache 22, and program module 24. In general, memory 16 can include any suitable volatile or non-volatile computer readable storage media. Cache 22 is a fast memory that enhances the performance of processing unit 14 by holding recently accessed data, and data near recently accessed data, from memory 16. As will be further depicted and described below, memory 16 may include at least one of program module 24 that is configured to carry out the functions of embodiments of the invention.

The program/utility (e.g., medical scan prioritization engine 104), having at least one program module 24, may be stored in memory 16 by way of example, and not limiting, as well as an operating system, one or more application programs, other program modules, and program data. Each of the operating systems, one or more application programs, other program modules, and program data or some combination thereof, may include an implementation of a networking environment. Program module 24 generally carries out the functions and/or methodologies of embodiments of the invention, as described herein.

Program instructions and data used to practice embodiments of the present invention may be stored in persistent storage 18 and in memory 16 for execution by one or more of the respective processing unit 14 via cache 22. In an embodiment, persistent storage 18 includes a magnetic hard disk drive. Alternatively, or in addition to a magnetic hard disk drive, persistent storage 18 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Network adaptor 28, in these examples, provides for communications with other data processing systems or devices. In these examples, network adaptor 28 includes one or more network interface cards. Network adaptor 28 may provide communications through the use of either or both physical and wireless communications links. Program instructions and data used to practice embodiments of the present invention may be downloaded to persistent storage 18 through network adaptor 28.

I/O interface(s) 26 allows for input and output of data with other devices that may be connected to each computer system. For example, I/O interface 26 may provide a connection to external devices 30 such as a keyboard, keypad, a touch screen, and/or some other suitable input device. External devices 30 can also include portable computer readable storage media such as, for example, thumb drives, portable optical or magnetic disks, and memory cards. Software and data used to practice embodiments of the present invention can be stored on such portable computer readable storage media and can be loaded onto persistent storage 18 via I/O interface(s) 26. I/O interface(s) 26 also connect to display 32.

Display 32 provides a mechanism to display data to a user and may be, for example, a computer monitor, touchscreen, or virtual graphical user interface.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 5, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 40 and cloud computing environment 50 can communicate with any type of computerized device over any type of network and/or network addressable connection (e.g., using a web browser).

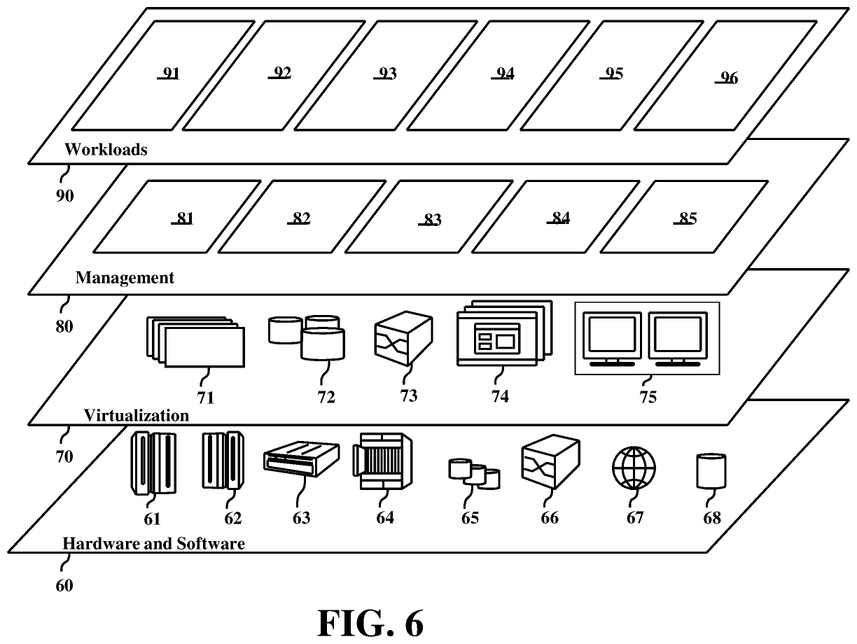

Referring now to FIG. 6, a set of functional abstraction layers provided by cloud computing environment 50 (depicted in FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 include hardware and software components. Examples of hardware components include mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include mapping and navigation 91; software development and lifecycle management 92; virtual classroom education delivery 93; data analytics processing 94; transaction processing 95; and medical scan review prioritization 96.

It should be noted that the embodiments of the present invention may operate with a user's permission. Any data may be gathered, stored, analyzed, etc., with a user's consent. In various configurations, at least some of the embodiments of the present invention are implemented into an opt-in application, plug-in, etc., as would be understood by one having ordinary skill in the art upon reading the present disclosure.

What is claimed is:

1. A computer-implemented method for prioritizing the review of medical scans in a medical record review system, the method comprising:
analyzing, by a processor, one or more medical scans each associated with a patient from a plurality of patients, based on a machine learning algorithm;
identifying, by the processor, at least one abnormal condition on the one or more medical scans, based at least in part on the analysis;
responsive to identifying at least one abnormal condition, prioritizing, by the processor, the at least one identified abnormal condition, wherein the abnormal condition is an urgent condition, based at least in part on the machine learning algorithm; and
presenting, by the processor, a scaled down version of the medical scan in a mini viewer window within the medical record review system.

2. The computer-implemented method of claim 1, wherein presenting a scaled down version of the medical scan in a mini viewer window further comprises:
preloading, by the processor, the full scale scan of the prioritized identified abnormal condition.

3. The computer-implemented method of claim 1, further comprising:
receiving, by the processor, a medical report associated with the prioritized scan; and
responsive to receiving a medical report, updating, by the processor, the machine learning algorithm, based on the received medical report.

4. The computer-implemented method of claim 1, wherein prioritizing the at least one identified abnormal condition further comprises:
scoring, by the processor, each of the medical scans with an identified abnormal condition, based at least in part on the identified abnormal condition;
generating, by the processor, an urgency score based, at least in part, on a scored medical scan and a health record of the patient associated with the scored medical scan; and
ranking, by the processor, the generated urgency score.

5. The computer-implemented method of claim 1, wherein the electronic medical record review system comprises:
a main window comprised of a plurality of assigned patient medical scans;
the mini viewer window comprised of a plurality of scaled down prioritized scans with one or more identified abnormal conditions; and
an input field, wherein the input field can receive a medical report.

6. The computer-implemented method of claim 1, wherein the machine learning algorithm comprises:
a convolutional neural network trained to identify one or more abnormalities in a medical scan.

7. The computer-implemented method of claim 3, wherein the machine learning algorithm comprises:
a natural language processing algorithm trained to identify medical terminology within the medical report and associate the medical scan with one or more conditions from within the medical report.

8. A computer system for prioritizing the review of medical scans in a medical record review system, the system comprising:
one or more computer processors;
one or more computer readable storage devices; and
computer program instructions to:
analyze one or more medical scans each associated with a patient from a plurality of patients, based on a machine learning algorithm;
identify at least one abnormal condition on the one or more medical scans, based at least in part on the analysis;
responsive to identifying at least one abnormal condition, prioritize the at least one identified abnormal condition, wherein the abnormal condition is an urgent condition, based at least in part on the machine learning algorithm; and
present a scaled down version of the medical scan in a mini viewer window within the medical record review system.

9. The computer system of claim 8, wherein presenting a scaled down version of the medical scan in a mini viewer window further comprises:
preload the full scale scan of the prioritized identified abnormal condition.

10. The computer system of claim 8, further comprising:
receive a medical report associated with the prioritized scan; and
responsive to receiving a medical report, update the machine learning algorithm, based on the received medical report.

11. The computer system of claim 8, wherein prioritizing the at least one identified abnormal condition further comprises:
score each of the medical scans with an identified abnormal condition, based at least in part on the identified abnormal condition;
generate an urgency score based, at least in part, on a scored medical scan and a health record of the patient associated with the scored medical scan; and
rank the generated urgency score.

12. The computer system of claim 8, wherein the electronic medical record review system comprises:
a main window comprised of a plurality of assigned patient medical scans;
the mini viewer window comprised of a plurality of scaled down prioritized scans with one or more identified abnormal conditions; and
an input field, wherein the input field can receive a medical report.

13. The computer system of claim 8, wherein the machine learning algorithm comprises:
a convolutional neural network trained to identify one or more abnormalities in a medical scan.

14. The computer system of claim 10, wherein the machine learning algorithm comprises:
a natural language processing algorithm trained to identify medical terminology within the medical report and associate the medical scan with one or more conditions from within the medical report.

15. A computer program product for prioritizing the review of medical scans in a medical record review system, the computer program product comprising:
a computer readable storage medium having program instructions embodied therewith, the program instructions executable by a processor to cause the processors to perform a function, the function comprising:
analyze one or more medical scans each associated with a patient from a plurality of patients, based on a machine learning algorithm;
identify at least one abnormal condition on the one or more medical scans, based at least in part on the analysis;
responsive to identifying at least one abnormal condition, prioritize the at least one identified abnormal condition, wherein the abnormal condition is an urgent condition, based at least in part on the machine learning algorithm; and
present a scaled down version of the medical scan in a mini viewer window within the medical record review system.

16. The computer program product of claim 15, wherein presenting a scaled down version of the medical scan in a mini viewer window further comprises:
preload the full scale scan of the prioritized identified abnormal condition.

17. The computer program product of claim 15, further comprising:
receive a medical report associated with the prioritized scan; and
responsive to receiving a medical report, update the machine learning algorithm, based on the received medical report.

18. The computer program product of claim 15, wherein prioritizing the at least one identified abnormal condition further comprises:
score each of the medical scans with an identified abnormal condition, based at least in part on the identified abnormal condition;
generate an urgency score based, at least in part, on a scored medical scan and a health record of the patient associated with the scored medical scan; and
rank the generated urgency score.

19. The computer program product of claim 15, wherein the electronic medical record review system comprises:
a main window comprised of a plurality of assigned patient medical scans;
the mini viewer window comprised of a plurality of scaled down prioritized scans with one or more identified abnormal conditions; and
an input field, wherein the input field can receive a medical report.

20. The computer program product of claim 15, wherein the machine learning algorithm comprises:
a convolutional neural network trained to identify one or more abnormalities in a medical scan.
